(19) United States
(12) Patent Application Publication
(10) Pub. No.: US 2008/0300499 A1
(43) Pub. Date: Dec. 4, 2008

(54) Portable Apnea and Cardiac Monitor

(76) Inventor: Richard Ellis Strube, Vestal, NY (US)

Correspondence Address: Richard E. Strube, 408 Elmhaven Dr., Vestal, NY, 13850, US

(21) Appl. No.: 12/122,936

(22) Filed: May 19, 2008

Related U.S. Application Data
(60) Provisional application No. 60/941,729, filed on Jun. 4, 2007.

(51) Int. Cl. A61B 5/0205

(52) U.S. Cl. 600/527; 600/534

(57) ABSTRACT

This invention relates to non-invasive monitoring devices, in monitoring minute movements made by the body to report the presence or absence of respiration and normal heart rate. The movements being measured are the pulsations made by the normal operation of both the heart and lungs. This is accomplished by converting the body movements into electrical signals with an adapted polymorph-piezoelectric transducer. The electrical signal that is detected is a mixed signal, a signal composed of a lower frequency (breathing contraction) and the higher frequency (heart pulse). The signals are detected by separating the body motions by an adaptive filter that has a break frequency at twice the frequency of the larger body movement signal (breathing contraction).

The separate body movement signals operate with associated logic circuitry to allow each signal to be independently measured, recorded, and acted upon to determine if the individual (patient) is undergoing a life threatening experience. Such an event by either signal will trigger an audio and visual display alarm which is attached on or near the patient. The alarm event trigger is also connected to a low voltage detector circuit to indicate that the supply voltage has dropped below an acceptable level.

### REFERENCES CITED [REFERENCED BY]

U.S. Patent Documents

| | | |
|---|---|---|
| 3545429 | December 1970 | Pelta et al. |
| 3584618 | June 1971 | Reinhard |
| 3608542 | September 1971 | Pacela et al. |
| 3643652 | February 1972 | Beltram |
| 3727606 | April 1973 | Sielaff |
| 3730173 | May 1973 | Deaton |
| 3782368 | January 1974 | Riebold |
| 3802417 | April 1974 | Lang |
| 3898981 | August 1975 | Basham |
| 4169462 | October 1979 | Strube |
| 4827924 | May 1989 | Japuntich |
| 5063938 | November 1991 | Beck et al. |
| 5383470 | January 1995 | Kolbly |
| 5857460 | January 1999 | Popitz |
| 6468222 | October 2002 | Mault et al. |
| 6895962 | May 2005 | Kullik et al. |
| 2002/0062830 | May 2002 | Meier et al. |
| 2003/0004427 | January 2003 | Swisa |
| 2004/0163648 | August 2004 | Burton |
| 2004/0233058 | November 2004 | Dodds |
| 2004/10/936992 | September 2004 | Caldwell |

Foreign Patent Documents

| | | |
|---|---|---|
| 1398752 | June, 1975 | GB |

### OTHER REFERENCES

“Respiration Monitor With Automatic Stimulation for Premature and Newborn Babies,” Fresenius Corp., West Germany, February 1974.
Chess, G. F. et al., “Apnea Monitor for Laboratory Animals,” Med. & Biol. Engr., v. 14, #1, pp. 97-100, January 1976.

### Primary Examiner:

### Assistant Examiner:

### BACKGROUND OF THE INVENTION

The problem of sudden unexpected death among children, especially infants, is unfortunately not a new phenomena and causes of these deaths are neither known nor are they fully understood. Similar phenomenon also exists for the elderly, many disabled and persons undergoing severe blunt force trauma in emergency situations. Fortunately though, for all of these differing types of patients there is a period of time between apparent death and permanent death during which if failure of respiration or abnormal rate of contraction of the heart can be detected, there remains enough time left to save the life.

Knowledge of how the body is reacting up to and during this episode is needed by medical personal in order to take appropriate measures. This device has onboard memory to record the condition prior to the episode with a last in last out (LILO) memory recorder. The memory can be downloaded to a standard PC for data manipulation via wireless connection while the sensor has been returned to its normal docking station or to a portable special purpose adapter for recharging of the power source.

The prior art presents a number of devices which can detect this pre-death situation. Typically though, these devices require either elaborate procedures, or controlled environments, or facility power, as in U.S. Pat. Nos. 3,643,652 “Medical Breathing Measuring System” by Delfin J. Beltran, or 3,730,173 “Stimulation Method and Apparatus for Attempting to Return a Physiological Parameter of a Patient to Normal” by David W. Deaton, or 3,898,981 “Respiration Monitoring Apparatus” by Raymon B. Basham, or 3,545,429 “Respiration Monitor” by Edmond R. Pelta, and others.

A device for detecting (or monitoring) the pulse of a patient is extremely important in emergency situations. This device is an alarm to allow others to find the individual needing assistance and extract them to safety for further treatment. As will be appreciated, in mass casualty situations such as those during a national emergency, individual alarms indicating the lack of movement allow a responder to immediately determine which individuals are in critical condition and need immediately attention.

### FIELD OF THE INVENTION

The present invention relates to devices for detecting or monitoring of vital bodily functions of a patient. More particularly, the present invention relates to a device for detecting or monitoring the heart rate and respiration of a patient.

### SUMMARY OF THE INVENTION

Because the most rigorous size wise design requirement is for the apparatus to be attached to an infant, this requires that everything associated with the project be miniaturized. Therefore, the design criteria used was that the detector must be:
(A) small enough to be attached to an infant;
(B) contain its own power source;
(C) not being dependent on an external environment; and
(D) not be overly affected by the moisture/bodily fluids found around infants and injured persons.
   To incorporate these criteria, the project broke into seven (7) major areas:
   (A) a self contained power source;
   (B) a charging means for the self contained power source;
   (C) an electromechanical transducer;
   (D) a logic or computational package;
   (E) a means of temporarily recording the information obtained by said computational package;
   (F) a means of transmitting the information recorded to long term storage/tracking/data manipulation means, and
   (G) an audio warning device.

It is accordingly an object of the invention to provide a motion detection method which overcomes the above-mentioned disadvantages of the heretofore-known devices and methods of this general type and which provides for a robust method of determining heart rate and respiration.

According another aspect of the invention, the device is configured to send a signal to a remote location to track aspects of heart rate and respiration; with the use of an adapter this information can be done continuously while the patient is being monitored.

### BRIEF DESCRIPTION OF THE DRAWING

FIG. 1 Is The Preferred Attachment Of Sensor To Patient.

FIG. 2 Is A Conception Sketch In Accordance With The Invention.

FIG. 3 Is A Functional Diagram Of The Motion Detector Monitoring System In Accordance With The Invention.—Sensor Detector Interface and Signal Conditioning

FIG. 4 Is A Functional Diagram Of The Motion Detector Monitoring System In Accordance With The Invention.—Detector Computational Logic

FIG. 5 Is A Functional Diagram Of The Motion Detector Monitoring System In Accordance With The Invention.—Detector-Base Station Interface and Connection to Outside Computer

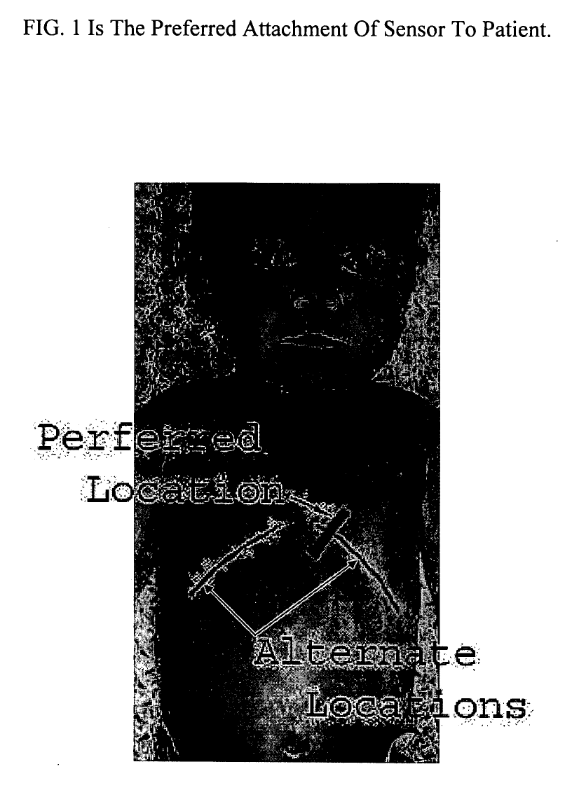

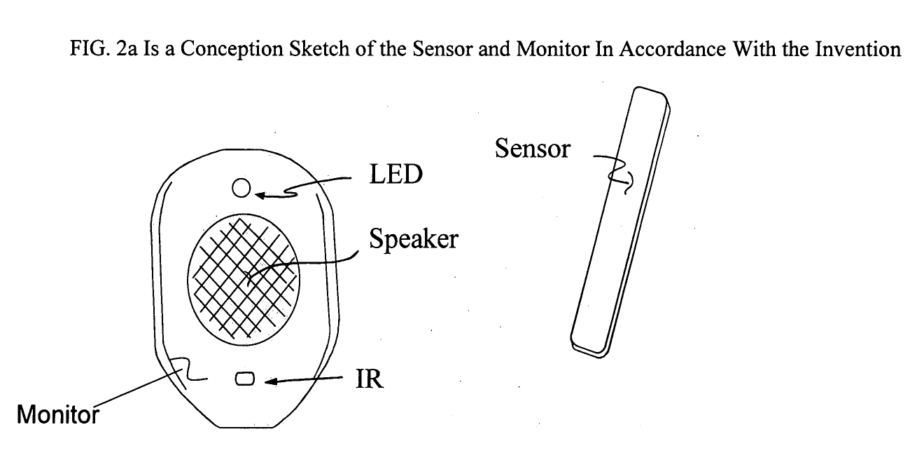

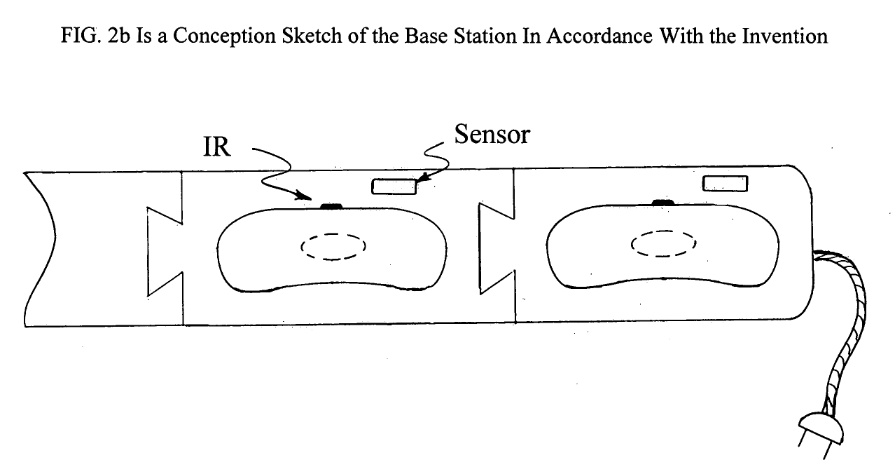

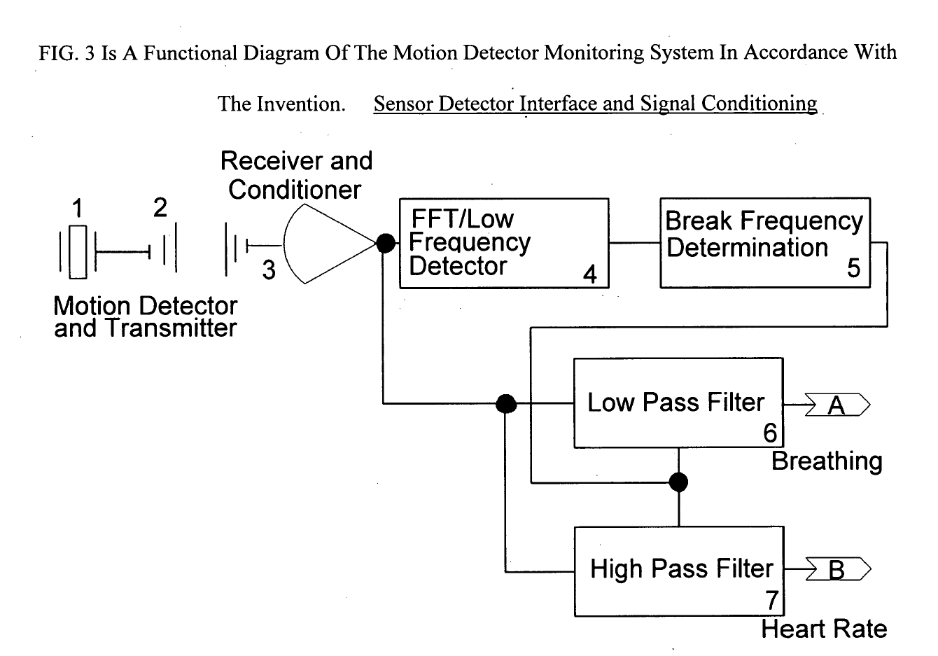

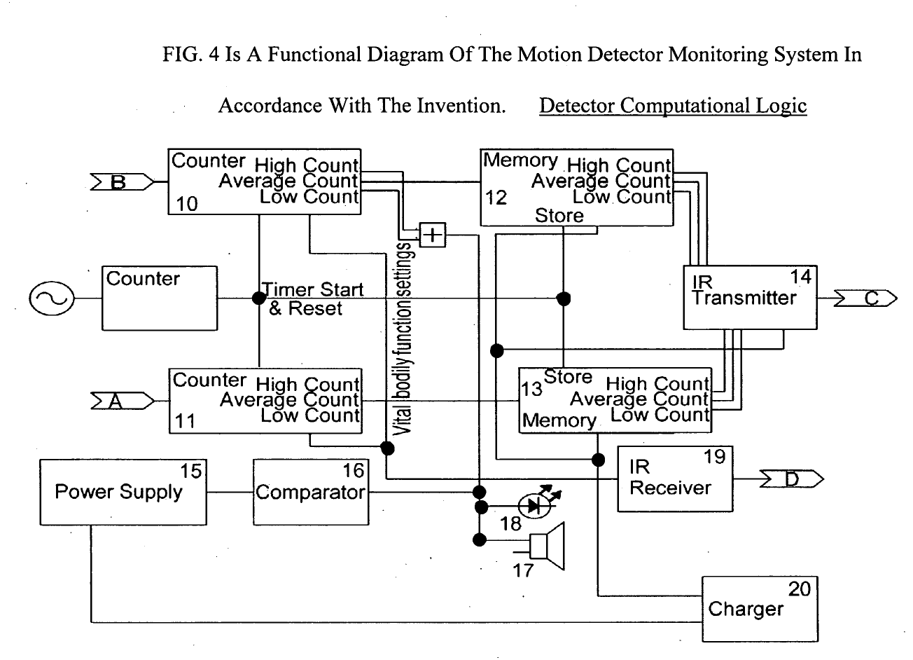

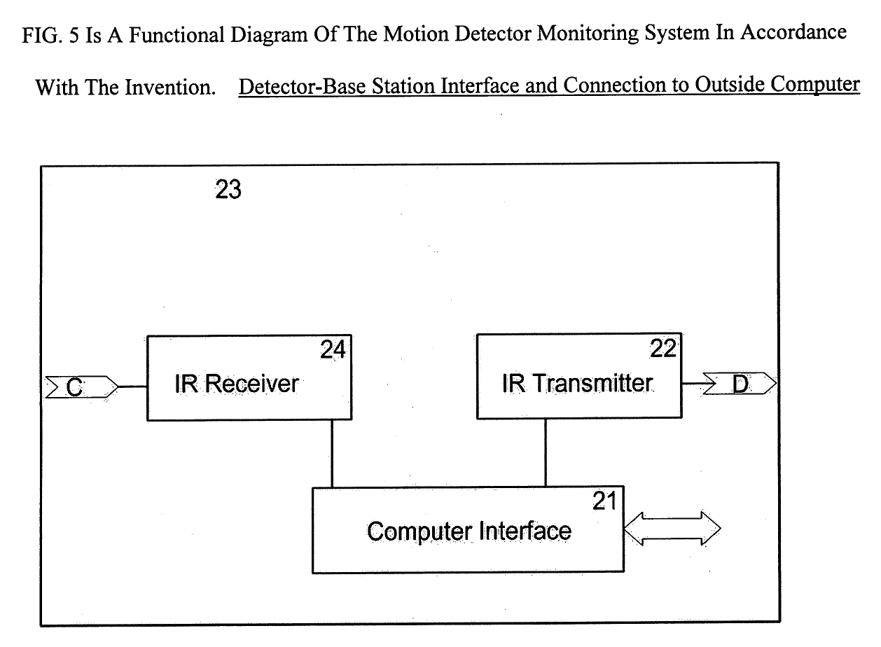

### DETAILED DESCRIPTION OF THE INVENTION

The circuitry of the apparatus is contained in custom integrated circuits. These integrated circuits consists of signal conditioners, FFT and low frequency detector, break frequency computational determination, low pass filter, high pass filter, oscillator counters, memory circuits, comparators, gating circuits, transmitters, receivers, audio transducer, power supply and charger circuit.

The signal originates from an impulse derived from mechanical pressure, the result of breathing and heart pulsations on the polymorph-piezoelectric transducer crystal (1). The impulse from the polymorph-piezoelectric transducer crystal is properly shaped and level translated by signal conditioners (2 and 3), which also act as transceivers.

The output of signal conditioner (3) if fed to a FFT/Low Frequency Detector (4) and a low pass filter (6) as well as a high pass filter (7).

The output of the FFT/Low Frequency detector (4) goes into the Break Frequency Determination circuit (5).

The output of the Break Frequency Determination circuit (5) goes to the low pass filter (6) and the high pass filter (7).

The output of the low pass filter (6) goes to the respiration rate counter (11) and if the counter circuit is zero for that period the audio transducer (17) is energized as well as a LED (18) visual indicator.

The output of the high pass filter (7) goes to the heart rate counter (10) and if the counter circuit is outside of acceptable range for that period the audio transducer (17) is energized as well as a LED (18) visual indicator.

The oscillator circuit (8) consists of a crystal controlled free running Schmitt-trigger oscillator for high stability. This oscillator along with its associated circuitry is used as the input to the 30 second counter circuit (9).

The 30 second counter circuit (9) is used as a set/reset for the heart rate (10) and respiration (11) counters and as timing inputs to the memory circuits (12 and 13) to take the counts and determine the high count, low count and average count for this period as compared with the previous periods.

The power supply (15) supplies power to all of the components and feeds the voltage comparator (16). The voltage comparator (16) determines when the power supply has insufficient charge to continue operation and feeds a pulsating signal to the audio transducer (17) is energized as well as a LED (18) visual indicator.

The charger circuitry (20) is in the base station (23) which also houses the computer interface (21). The computer interface is to allow for custom settings for heart rate and respiration to be uploaded to the monitor to adjust for patient age, weight or other considerations. The computer interface (21) communicates through an IR transmitter (22). The IR transmitter (22) communicates with the monitor mounted IR receiver (19) which sets the limit values in the respiration rate counter (11) and the heart rate counter (10).

Note that the base station (23) can be replaced by a wearable attachable station that acts as a charger circuit for the power supply (15) and as a continuous counter down loader for the heart rate (10) and respiration (11) counters for communication with a computational device.

What is claimed is:

1. A portable motion detector for monitoring the heart rate and respirational activity of a patient, which comprises; polymorph-piezoelectric transducer means adapted for converting body expansion during the heart pumping and breathing process into a mixed electrical signal,

2. In a digital signal processing system, a method of computing a motion decision value from an electromechanical transducer which comprises the following steps:
(A) A signal de-mixing method of splitting the bodily motion signal into a high pass signal (heart rate) and a low pass signal (breathing/respiration);
(B) a logic or computational package;
(C) a means of temporarily recording the information obtained by said computational package;
(D) a means of transmitting the information recorded to long term storage/tracking/data manipulation means, and
(E) an audio and visual warning device,
(F) a self contained power source;
(G) a charging means for the self contained power source;

3. The method according to claim 2, which further separates the electrical signal by using low-pass/high-pass filtering de-multiplexation of the composite signal into separate signals by an adaptive filter, a lower frequency (breathing/respiration contraction) and a higher frequency (heart pulse). The adaptive filter that has a break frequency at twice the frequency of the larger body movement signal (breathing/respiration contraction).

4. The method according to claim 2, wherein the step of forming the motion detection signal comprises computing where the separate body movement signals operate with associated logic circuitry to allow each signal to be independently measured, recorded, and acted upon to determine if the individual (patient) is undergoing a life threatening experience.

5. The method according to claim 2, wherein the step of forming the motion detection signal comprises separating each bodily motion signal where:
(A) The counting circuit coupled to the output of said signal de-mixing means for producing an output signal representative of said heart pumping signal being not within an except-able limit over a predetermined counting period,
(B) counting circuit coupled to the output of said signal mixing means for producing an output signal representative of the absence of a pumping or chest movement breathing signal over a predetermined counting period,
(C) an oscillator circuit coupled to the input of said counting circuit for providing a signal for counting,

6. The method according to claim 4, wherein the output of said counting circuits for producing a signal indicating that heart rate is not within an acceptable limit or that respiration has ceased will trigger an audio and visual display alarm which is attached on or near the patient.

7. The method according to claim 2, wherein the low voltage detector circuit means coupled to the input of said audio output means for producing an output indicative of supply voltage dropping below a predetermined level, switch means coupled to the output of said oscillator circuit and to the output of said low voltage detection circuit means for enabling output of said low voltage detection circuit means whereby a pulsating and visual tone signal indicative of a low battery power is produced.

8. An apparatus as in claim one (5) the said oscillator is a Schmitt-trigger oscillator.

9. An apparatus as in claim one (5) the said oscillator is a variable oscillator.

10. An apparatus as in claim one (6) the said visual display alarm is an LED.

11. An apparatus as in claim one (6) the said audio alarm is a piezoelectric transducer.
